(19) United States
(12) Patent Application Publication
(10) Pub. No.: US 2009/0149800 A1
(43) Pub. Date: Jun. 11, 2009

(54) IONTOPHORETIC DRUG DELIVERY DEVICE AND SOFTWARE APPLICATION

(75) Inventor: Emma Amelia Durand, Jamestown, RI (US)

Correspondence Address: PILLSBURY WINTHROP SHAW PITTMAN, LLP, P.O. BOX 10500, MCLEAN, VA, 22102, US

(73) Assignee: Isis Biopolymer LLC, Warwick, RI (US)

(21) Appl. No.: 12/330,698

(22) Filed: Dec. 9, 2008

Related U.S. Application Data
(60) Provisional application No. 61/012,582, filed on Dec. 10, 2007.

(51) Int. Cl. A61N 1/30; G06F 17/00; G06F 9/44

(52) U.S. Cl. 604/20; 700/90; 717/109

(57) ABSTRACT

The iontophoretic drug delivery system includes electrodes controlled by a microprocessor controller to drive charged molecules contained in a drug reservoir through the skin into the issues of a patient. The iontophoretic drug delivery system further includes an antenna connected to the programmable microprocessor. The antenna allows for the programming of the microprocessor and for the exchange of patient, drug, and treatment related information between the microprocessor and an external device. The iontophoretic drug delivery system is also provided with buttons to allow a patient to manually activate the drug delivery system. The iontophoretic drug delivery system is housed within a thin polyester film membrane.

This patent application claims priority to provisional application 61/012,582 filed on Dec. 10, 2007 entitled Iontophoretic Drug Delivery Device and Software Application by Inventor Emma Amelia Durand, the contents of which is incorporated by reference herein in its entirety.

### FIELD OF THE INVENTION

The present invention relates to the field of devices and systems for delivering drugs to medicate a patient, and more particularly to an iontophoretic drug delivery system.

### BACKGROUND OF THE INVENTION

Iontophoresis is a drug delivery system. Iontophoresis is a non-invasive method of propelling charged molecules, normally medication or bioactive-agents, transdermally by repulsive electromotive force. By applying a low-level electrical current to a similarly charged drug solution, iontophoresis repels the drug ions through the skin to the underlying tissue. In contrast to passive transdermal patch drug delivery, iontophoresis is an active (electrically driven) method that allows the delivery of soluble ionic drugs that are not effectively absorbed through the skin.

An electrode drives charged molecules into the skin. Drug molecules with a positive charge are driven into the skin by an anode and those molecules with a negative charge are driven into the skin by a cathode.

There are a number of factors that influence iontophoretic transport including skin pH, drug concentration and characteristics, ionic competition, molecular size, current, voltage, time applied and skin resistance. Drugs typically permeate the skin via appendageal pores, including hair follicles and sweat glands.

Iontophoresis has numerous advantages over other drug delivery methods. The risk of infection is reduced because iontophoresis is non-invasive. Also, iontophoresis provides a relatively pain-free option for patients who are reluctant or unable to receive injections. For skin tissues, drug solutions may be delivered directly to the treatment site without the disadvantages of injections or orally administered drugs. Further, iontophoresis minimizes the potential for further tissue trauma that can occur with increased pressure from an injection.

### SUMMARY OF THE INVENTION

An iontophoretic drug delivery system is disclosed. The iontophoretic drug delivery system includes electrodes controlled by a microprocessor controller to drive charged molecules through the skin into the tissues of a patient. The iontophoretic drug delivery system further includes a wireless signal receiver connected to the microprocessor controller. The wireless signal receiver allows for the programming of the microprocessor and for the exchange of patient, drug, and treatment related information between the microprocessor and an external device. The microprocessor may be programmed through the wireless signal receiver with drug delivery schedule information, including frequency and dosage, for a particular patient and medication. A drug reservoir contains charged drug molecules that are driven into the skin by the electrodes. The operation of the electrodes, frequency, duration, and level of voltage applied, is controlled by the microprocessor. A battery provides power to the iontophoretic device.

The iontophoretic drug delivery system may be optionally housed within a thin polyester film membrane. The iontophoretic drug delivery system is configured in the shape of a generally flexible patch that adheres to the skin of a patient with an adhesive. In one embodiment, the edges of the flexible patch may be provided with a high tack adhesive to maintain the integrity of the skin-patch boundary. A lower tack adhesive is provided within the internal area of the flexible patch to make the purposeful removal of the patch from the use less painful. The drug reservoirs can be formed of a membrane or a gel pad in which charged drug particles are injected.

The iontophoretic drug delivery system may contain different various numbers of drug reservoirs depending upon the particular treatment. Where a single drug is being delivered, the system may contain a single drug reservoir adjacent one electrode. Where a treatment requires two drugs that have oppositely charged solutions, the system may include a reservoir adjacent each of the oppositely charged electrodes. Where multiple drugs having the same charge are used, they may be either mixed into a single drug reservoir or placed in multiple drug reservoirs each adjacent a respective electrode having the same electric charge.

The size of the electrodes may vary in different embodiments depending upon the strength of the electrical current needed to be produced in order to drive drug molecules of various sizes into a patient's skin.

In one exemplary embodiment, the electrodes and the microprocessor, battery and antenna are attached on opposite sides of a flexible sheet. The electrodes, microprocessor, battery and antenna are electrically connected utilizing conductive silver ink. Through holes formed in the flexible sheet electrically connect the electrodes to the microprocessor, battery and antenna. The microprocessor and battery are attached to the system using conductive cement.

In another embodiment, the system main contain various sensors to measure parameters such as patient skin temperature, moisture at the system/patient skin interface, or other patient or drug delivery related parameters.

In another embodiment, the system includes a software module for creating a set of dosage instructions for the microprocessor to control the operation of the electrode to administer the charged drug molecules held in the drug reservoir. A programming device is provided for communicating the dosage instructions to the microprocessor through the wireless signal receiver. The dosage instructions can include duration information for turning the electrode ON and OFF. The dosage instructions can also include voltage information for a level of voltage placed across the electrode when turned ON. The dosage instructions may be selected from a database based upon a type of the charged drug molecules and a patient parameter. The dosage instructions may also be created manually by a user using the software module.

Other objects, features and aspects of the invention will become apparent from the following detailed description, the accompanying drawings, and the appended claims.

### BRIEF DESCRIPTION OF THE DRAWINGS

The novel features that are considered characteristic of the invention are set forth with particularity in the appended claims. The invention itself; however, both as to its structure and operation together with the additional objects and advantages thereof are best understood through the following description of the preferred embodiment of the present invention when read in conjunction with the accompanying drawings, wherein:

FIG. 1 discloses an exploded isometric view of a iontophoretic drug delivery system;

FIG. 2 discloses an isometric view of an iontophoretic drug delivery system;

FIG. 3 discloses an isometric see-through view of an iontophoretic drug delivery system;

FIGS. 4-14 disclose a process of forming circuitry for an iontophoretic drug delivery system, wherein:

FIGS. 4 and 4A depict a printing of circuitry on a primary component side of a layer;

FIG. 5 depicts a deposition of dielectric material on a primary component side of a layer;

FIG. 6 depicts a printing of circuitry on a secondary component side of a layer;

FIG. 7 depicts formation of electrodes on a secondary component side of a layer;

FIG. 8 depicts a deposition of dielectric material on a secondary component side of a layer;

FIG. 9 depicts a filing of a through hole in a layer;

FIG. 10 depicts the attachment of laser cut foam to a secondary component side of a layer;

FIG. 11 depicts a formation of drug reservoirs on a secondary component side of a layer;

FIG. 12 depicts a deposition of a conductive epoxy on a primary component side of a layer;

FIG. 13 depicts a placement of components on a primary component side of a layer;

FIG. 14 depicts a deposition of an encapsulant on a primary component side of a layer;

FIG. 15 illustrates a completed primary component side of a layer;

FIG. 16 illustrates a completed secondary component side of a layer;

FIG. 17 illustrates a side view of an iontophoretic drug delivery system;

FIG. 18 illustrates an adhesive pattern on a secondary component side of a layer;

FIG. 19 illustrates an iontophoretic drug delivery system having three drug reservoirs; and

FIG. 20 illustrates a side view of a button for manually operating an iontophoretic drug delivery system.

FIG. 21 illustrates a block diagram of a network system for prescribing medication and programming an iontophoretic drug delivery system;

FIG. 22 illustrates a flow chart depicting a processing for prescribing medication and programming an iontophoretic drug delivery system;

FIG. 23 illustrates a software module diagram of the software for prescribing medication and programming an iontophoretic drug delivery system;

FIG. 24 illustrates a screen shot of a patient profile software module;

FIG. 25 illustrates a screen shot of a prescription information software module;

FIG. 26 illustrates a screen shot of a patient information software module having a default prescription profile;

FIG. 27 illustrates a screen shot of a manual level adjustment module;

FIG. 28 illustrates a screen shot of a confirmation software module;

FIG. 29 illustrates a screen shot of a prescription module for printing prescription labels and for programming an iontophoretic drug delivery system; and

FIG. 30 illustrates an isometric view of a iontophoretic drug delivery system being wirelessly programmed by the network system.

### DETAILED DESCRIPTION OF PREFERRED EMBODIMENTS

While the invention has been shown and described with reference to a particular embodiment thereof, it will be understood to those skilled in the art, that various changes in form and details may be made therein without departing from the spirit and scope of the invention.

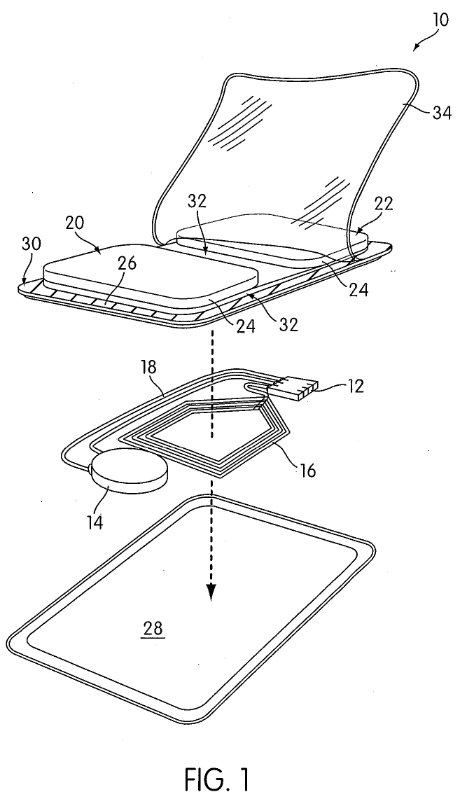

FIG. 1 discloses an exploded isometric view of an iontophoretic drug delivery system 10. System 10 provides a non-invasive method of propelling high concentrations of a charged substance, normally medication or bioactive-agents, transdermally by repulsive electromotive force. Iontophoretic drug delivery system 10 includes a microprocessor controller 12, a battery 14, an antenna 16, printed flexible wiring 18, an electrode 20, and an electrode 22. Drug reservoirs 24 are coupled to electrodes 20 and 22. Electrodes 20 and 22 and drug reservoirs 24 are contained in flexible layer 26 that conforms to the patient's body in the area of application. Layer 26 and layer 28 are bonded together to seal and protect microprocessor controller 12, battery 14, antenna 16, and printed flexible wiring 18. The construction and configuration shown is an example and not intended to be limiting.

Antenna 16 provides a wireless capability for system 10 to communicate with other external devices. In an exemplary embodiment, antenna 16 may be an RFID antenna, a blue-tooth enabled device, an infra-red wireless device, or another wireless signal receiver. Antenna 16 may function as an RFID antenna or can receive signals from an outside device through capacitive coupling. Antenna 16 can also be configured in the shape of inductive coils in order to receive signals from an outside device through inductive coupling.

A high-tack adhesive 30 is placed on an outer edge of layer 26 and a low-tack adhesive 32 is placed within the internal area of the skin contacting surface of layer 26. High-tack adhesive 30 extends around the periphery of layer 26 and secures the outer edge of system 10 to the skin of a patient. High-tack adhesive 30 is used to prevent moisture or physical force from peeling system 10 off of the skin of a patient. Low-tack adhesive 32 is placed in the internal area of layer 26 (i.e. inward with respect to the high tack adhesive 30) to maintain contact between system 10 and the skin of the patient. The use of low-tack adhesive 32 makes removal of system 10 from the skin of a patient less painful, while the high tack adhesive 30 provides stronger bonding at the periphery where it is needed most to prevent lifting of the edge of system 10 or exposing system 10 to moisture. A preferred type of adhesive for high-tack adhesive 30 is a silicone based adhesive that is rapidly cured with an electron beam or UV radiation. Preferably, the adhesive is not present between the drug reservoir 24 and the skin, as this contact could alter the properties of adhesive 30 and/or influence the release of the drug. System 10 eliminates any interaction between the drug and adhesive matrix. In an exemplary embodiment, these adhesives may have peel strengths of 8.5 or 9.3 lbs/in. Adhesives with stronger or weaker peel strengths may be used with system 10.

A release layer 34 is placed over adhesive 30 and 32 to protect adhesive 30 and 32. Layer 34 is removed from system 10 just prior to bonding system 10 to the skin of a patient. Layer 34 makes sufficient contact with adhesive 30 and 32 to hold layer 34 to system 10 while allowing a user to easily peel layer 34 off of system 10. Typically, layer 34 is coated with a silicone based release coating to ensure that it can be peeled off without degrading adhesives 30 and 32.

Charged drug molecules are contained within drug reservoirs 24, which faces the patient's skin through an opening in layer 26. Drug reservoirs 24 may be a gel pad or membrane to which the charged drug molecules contained in a solution are applied or injected. By impregnating a gel pad or membrane with charged drug molecules, the charged drug molecules are not able to readily be absorbed into a patient's body without the operation of electrodes 20 and 22. In one embodiment, drug reservoirs 24 are a conductive medium to support the function of electrodes 20 and 22. By making drug reservoirs 24 also a conductive medium, system 10 can function with a lower amount of current, thereby extending battery 14 life and reducing the amount of current put into a patient's skin, of which a high amount of current can cause irritation. Typically, the solution is injected through a port into drug reservoirs 24. Electrodes 20 and 22 drive the charged drug molecules out of drug reservoirs 24 into the skin of a patient. Where the reservoir 24 includes a gel, the drug in ionic form may be mixed with the gel matrix cured together and assembled into the system 10.

The basis of ion transfer lies in the principle that like poles repels and unlike poles attract. Ions, being particles with a positive or a negative charge are repelled into the skin by an identical charge the electrode places over it. When a direct electric current activates electrodes 20 and 22, anions in the solution, ions with a negative charge, are repelled from the negatively charged electrode. Positively charged ions (cations) are likewise repelled from the positive electrode. The electrical current drives ions through the skin that would not be absorbed passively. The quantity of ions that are made to cross the skin barrier is proportional to the current density and to the amount of time the current flows through the solution. Current density is determined by the strength of electric field and the electrode size. A desired current strength is in the range of 0.4 mA or 2.0 mA per square inch of electrode 20 and 22 surface. This current strength is below sensory perception of a typical human patient. If electrodes 20 and 22 are too small, thereby concentrating the current (or if the current is too high), it may be more uncomfortable for the patient, as the current density may be sensed as an irritant.

Electrodes 20 and 22 and flexible printed wiring 18 are preferably made from a flexible material that can bend with layer 26 in conformity to the application area of the patient's body. One exemplary flexible material is silver conductive ink with resistivity of 8 to 10 milliohms per square. The resistivity of silver conductive ink within the range of 8 to 10 milliohms per square is desirable in order to have sufficient current to drive drugs into the stratum corneum. The ink may be silver (Ag), for example, and may be printed (e.g. by screen printing or gravure rolling) onto layer 26. Most commercially available silver conductive inks have a resistivity in the range of 14 to 18 milliohms per square, which limits the current available to drive the drugs through the stratum corneum. Electrodes 20 and 22 may be formed of silver chloride (AgCl).

System 10 includes two electrodes 20 and 22. In a particular drug treatment, the charged drug molecules will typically have one charge. Thus, only one of electrodes 20 or 22 can drive the charged drug molecules into the skin of the patient. The electrode that drives the charged drug molecules into the patient's skin is sometimes referred to as an active electrode, which is coupled with drug reservoir 24. A passive electrode that is not coupled to a drug reservoir 24 completes the circuit with the active electrode for creating a current for driving charged drug molecules into the patient's skin. In other drug treatments, the solutions containing charged drug molecules may have both positive and negative charges. In that example, both electrodes are active electrodes and both are coupled to a drug reservoir 24.

In many drug treatments, a single drug is used. However, it is common for the efficacy of many drugs to be increased by combining their delivery with other drugs. Thus, system 10 may be configured to deliver multiple types of charged drug molecules. In the case where the multiple drug molecules have the same charge, those drugs may be combined into a single solution and delivered from a single drug reservoir 24. In other embodiments where the multiple drugs have the same charge, but need to be delivered to the patient at different times or in different quantities, multiple electrodes 22 with multiple drug reservoirs 24 may be used. In a case where there are two drugs having molecules of opposite polarity, both electrodes 20 and 22 are provided with drug reservoirs 24 for delivering their respective drugs to the patient. In one embodiment, drug reservoirs 24 are formed of hydro-gel (i.e., a water-based gel). In another embodiment, drug reservoirs 24 are formed on a membrane. The size electrodes 20 and 22 will vary depending upon the size of the charged drug molecule that they are trying to repel into the patient's skin. Thus, in embodiments where multiple electrodes with multiple drug chambers 24 are used, the sizes of the electrodes and drug chambers may vary,

One or both electrodes 20 and 22 are made of Ag/AgCl printable conductive ink coating. Electrodes 20 and 22 are covered by drug reservoirs 24, which may be formed from hydrogel that contains the charged drug molecules. Electrodes 20 and 22 are printed to the flexible printed wiring 18 with a highly conductive Polymer Thick Film (PTF) ink. In a preferred embodiment, a lead-free, silver loaded isotropic conductive cement is used that provides an electrical and mechanical connection having resistance to moisture and thermal shock.

Battery 14 powers system 10. It is desirable to make battery 14 as thin as possible, along with the rest of system 10, in order to enhance the ability of system 10 to adhere to a patient's skin with minimal disruption to the patient. Battery cells on the order of 0.7 mm thickness can generate up to 3.0 volts of electricity and multiple arrays can generate and control up to 9.0 volts of electricity. This amount of power allows for wireless programming and data acquisition with microprocessor controller 12 through antenna 16. The type and construction of the battery is not intended to be limiting.

Iontophoretic drug delivery system 10 may be used, in one exemplary embodiment, as a method of local drug delivery in a variety of clinical settings. System 10 can administer a local anesthetic to prevent painful sensations during skin puncture procedures, such as gaining venous access or injecting a drug intradernally or subcutaneously. System 10 can also deliver nonsteroidal anti-inflammatory drugs and corticosteroids in patients with musculoskeletal inflammatory conditions.

The rate, timing and pattern of drug delivery using iontophoretic drug delivery system 10 is controlled with microprocessor controller 12 by varying the electrical current applied to electrodes 20 and 22. Microprocessor controller 12 can be programmed to provide a variety of drug delivery profiles where the duration and frequency of drug delivery is varied based upon the treatment parameters. The speed with which a drug delivery system can provide efficacious blood levels of the target drug determines the onset of therapeutic action. Iontophoretic drug delivery system 10 allows many drugs to pass directly through the skin into underlying issue and the bloodstream at a rate that is significantly more rapid than oral or passive transdermal drug delivery methods. Microprocessor controller 12 is programmed wirelessly through antenna 16. In one exemplary embodiment, microprocessor controller 12 to configured accept programming once and only once, thereby ensuring that system 10 could not be erroneously reprogrammed or purposefully misprogrammed by various electronic devices.

As an option, microprocessor controller 12 may also perform the function of data acquisition of drug delivery information on the actual drug delivery performed by system 10. Drug delivery information, for example, can include an electronic record of the date, time and quantity of each dose delivered; providing information for determining patient compliance. Electrodes 20 and 22 can be used to determine whether system 10 is in contact with the patient's skin by the operation of electrodes 20 and 22 and the resistivity of the patient's skin in the electrode-skin-electrode circuit formed when system 10 is in contact with the patient's skin.

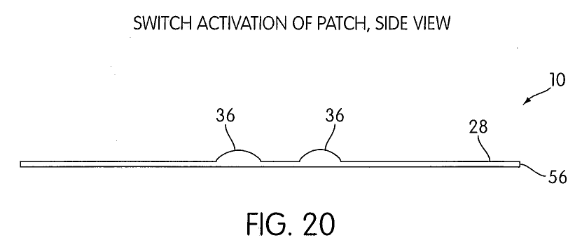

As an option, system 10 also may include a manual button array 36 (shown in FIG. 20). Manual button array 36 is coupled to microprocessor controller 12. Manual button array 36 allows a patient to manually operate system 10. System 10 is preferably programmed with drug delivery information to automatically deliver drugs to the patient. A patient can deviate from or override this program and manually operate system 10 to deliver drugs with manual button array 36. Manual button array 36 can allow a patient to deviate from the drug delivery information and provide either longer or shorter drug dosages more or less often than instructed in the drug delivery information. A patient can also turn off system 10 with manual button array 36, for example when they are feeling negative side affects from the drug delivery.

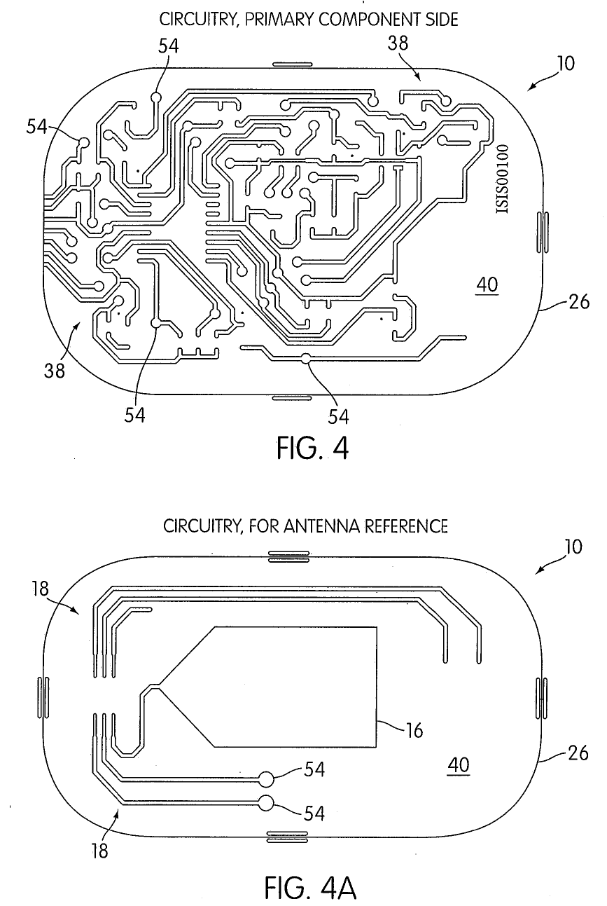

Electrodes 20 and 22, flexible printed wiring 18, antenna 16 and other circuitry components in system 10, in a preferred embodiment, are made from Polymer Thick Film (PTF) flexible circuits that are manufactured using a technology that consists of a low-cost polyester dielectric substrate and screen-printed thick film conductive inks. These circuits are made with an additive process involving the high-speed screen printing of conductive ink. Multi-layer circuits are manufactured using dielectric materials as an insulating layer, and double-sided circuits using printed through-hole technologies. FIGS. 4-15 show an exemplary method of fabricating system 10. Both active and passive surface mount components can be adhered to PTF flexible circuit assemblies with Conductive Adhesives (CA's) or with Anisotropic Conductive Adhesives (ACA's). In a preferred embodiment, to ensure optimal performance when system 10 is flexed, all components are encapsulated between layers 26 and 28, which are bonded together using a hydrophobic UV-cured material developed specifically for medical applications.

It is advantageous to utilize PTF flexible circuits because they are inherently less costly than for example copper based circuits. PTF are formed on a dielectric substrate that circuit traces are printed directly upon. In addition, PTF typically uses a PET substrate which is significantly less expensive than the polyimide substrate which is commonly used in copper circuitry. In addition, as PTF circuits are more environmentally friendly as they are printed directly and do not require the removal of materials where chemicals are used to selectively etch away the copper foil to leave behind a conductive pattern.

The charged drug molecules vary in size for different drug compounds. Larger drug molecules require stronger electromagnetic forces to drive them into the skin of a patient. Smaller drug molecules require lesser electromagnetic forces to drive them into the skin of a patient. Thus, it is desirable to vary the size of electrodes 20 and 22 based upon the size of the drug compounds in order to deliver an optimal amount of electromagnetic force to drive the drug molecules into the patient's skin. System 10 is therefore preferably manufactured for a specific drug molecule size by having a tailored size for each electrode 20 and 22.

The table shown below provides an exemplary list of drugs, the charge of the drug molecules and solution, and the purpose/condition for which the drugs are used.

| | Charge of | |
|---|---|---|
| | Solution/Drug | |
| Drug | Molecules | Purpose/Condition |
| Acetic acid | − | Calcium deposits |
| Atropine sulphate | + | Hyperhidrosis |
| Calcium | + | Myopathy, myospasm |
| Chloride | − | Sclerolytic, scar tissue |
| Citrate | − | Rheumatoid arthritis |
| Copper | + | Astringent |
| Dexamethasone | − | Tendinitis, bursitis |
| Glycopyrronium bromide | + | Hyperhidrosis |
| Iodine | − | Sclerolytic, scar tissue |
| Lidocaine | + | Dermal anesthesia |
| Magnesium | + | Muscle relaxant |
| Penicillin | − | Infected burn wounds |
| Poldine methyl sulfate | − | Hyperhidrosis |
| Potassium iodide | − | Scar Tissue |
| Salicylate | − | Analgesic, plantar warts |
| Sodium chloride | − | Scar tissue |
| Silver | + | Chronic osteomyelitis |
| Zinc | + | Antiseptic, wound healing |

In various embodiments, the flux of charged drug molecules from drug reservoirs 24 into the patient's skin can be increased through the use of a skin permeation enhancer. A permeation enhancer is any chemical or compound that, when used in conjunction with the charged drug molecule, increases the flux of charged drug molecules from drug reservoir 24 into the skin of the patient. That is, skin permeation enhancers is a substance that enhances the ability of the charged drug molecule transfer from the drug reservoir and permeate into the patient's skin.

Such use of a permeation enhancers is advantageous because it reduces the amount of electrical power required to transfer the drug from a reservoir 24 and into the patient's skin. This means that less current can be used, which in turn reduces the potential for skin irritation. And it also means less power is drawn, meaning the battery can be made smaller and/or last longer.

The enhancer may be an excipient, i.e., a medicinally inactive agent, included in the reservoir 24 with the charged drug molecule. Preferably, where a gel is used in the reservoir to carry the drug, the permeation enhancer and the drug are soluble in the gel but not chemically bonded to the gel network, thus enabling them to more easily transfer from the gel to the skin. In some embodiments, the enhancer may be a molecule with a charge similar to the associated drug molecule.

For example, oleic acid has an synergistic effect on the ability of iontophoresis to promote skin permeation of insulin. The use of propylene glycol further increased this effect. One exemplary incipient that can enhance the flux of charged drug molecules from system 10 into a patient by means of iontophoresis is a fatty acid having from 1-9 carbon atoms. Preferably, the incipient contains at least one C₂-C₆ fatty acid. By means of an example, the fatty acid may be selected from the group of propionic acid, valeric acid, 2-methylbutanoic acid, 3-methylbutanoic acid, and combinations thereof. In one example, the fatty acid is a mixture of propionic acid and valeric acid.

The permeation enhancer need not be in the reservoir 24 with the drug, and could be applied to the skin contacting surface of the reservoir 24. This could help create an interface between the reservoir 24 and the skin for enhancing permeation of the drug.

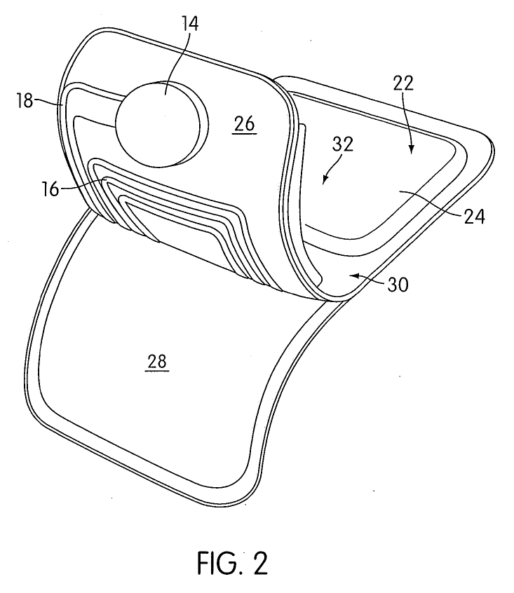

FIG. 2 discloses an isometric view of an iontophoretic drug delivery system 10. Battery 14, antenna 16, and flexible printed wiring 18 are shown adhered to layer 26 with layer 28 partially pealed away. FIG. 2 demonstrates the flexibility of system 10 that enables system 10 to conform to the contours of a patient's body and be able to deform during normal activity and movement of the patient's body. In addition, this figure shows how system 10, when assembled, is a thin patch that intrudes minimally upon the patient's daily functions.

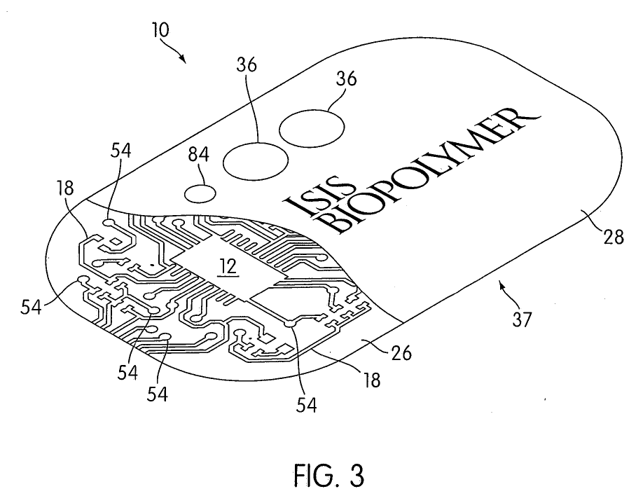

FIG. 3 discloses an isometric see-through view of an iontophoretic drug delivery system 10. Microprocessor controller 12, battery 14, antenna 16, printed flexible wiring 18, electrodes 20 and 22, and drug reservoirs 24 are shown sandwiched between layers 26 and 28. Manual button array 36 allows a patient to manually operate system 10. An indicator light 84 provides a visual indication of the status of system 10. Indicator light 84 is preferably a multi-colored LED, which may for example show green when operating normally, flash orange in a low power state, or flash red when a system failure occurs, as a non-limiting example. System 10 can include a variety of sensors 37 to monitor various parameters in the patient/system 10 environment. These parameters can include, by means of a non-limiting example, moisture, temperature, system 10/patient physical contact, and various patient parameters such as skin temperature, heart rate, etc. Information from sensors 37 can be used to provide positive feedback to system 10. For instance, if sensors 37 detect moisture at the system 10/patient skin interface, that may indicate that the patient is sweating. With this information, system 10 may be programmed to either increase the voltage delivered to electrodes 20 and 22 to drive the charged drug molecules through the added layer of sweat. Alternatively, system 10 may be programmed to stop delivery of the charged drug molecules until after the patient stops sweating and the sweat has evaporated.

FIGS. 4-14 disclose a process of forming circuitry for an iontophoretic drug delivery system 10. FIG. 4 depicts a printing of circuitry 38 on a primary component side 40 of layer 26. Layer 26 is preferably made of a thin flexible film, such as polyethylene terephthalate (PET). Circuitry 38 is made of conductive silver ink that is printed onto layer 26. In FIG. 4A, antenna 16 is printed along with wirings 18 that interconnect antenna 16, battery 14, and microprocessor controller 12.

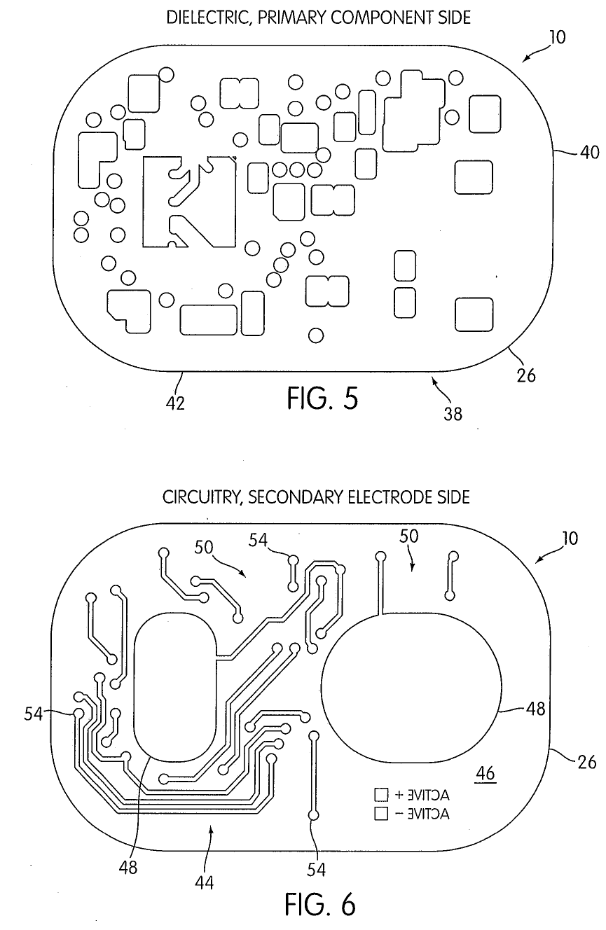

FIG. 5 depicts a deposition of dielectric material 42 on primary component side 40 of layer 26. Dielectric material 42 covers wirings 18 that interconnect antenna 16, battery 14, and microprocessor controller 12. Dielectric material 42 does not cover antenna 16. At this step, through holes 54 are formed by laser cutting layer 26. The dielectric material is printed on to layer 26. The dielectric is printed using a magnesium silicate pigment that is bound with urethane acrylate.

FIG. 6 depicts a printing of circuitry 44 on a secondary component side 46 of layer 26. Circuit 44 includes wirings 48 for electrodes 20 and 22 and wirings 50 for connecting electrodes 20 and 22 to battery 14 and microprocessor controller 12. Circuitry 44 is made of conductive silver ink that is printed onto layer 26. Secondary component side 46 makes contact with a patient's skin.

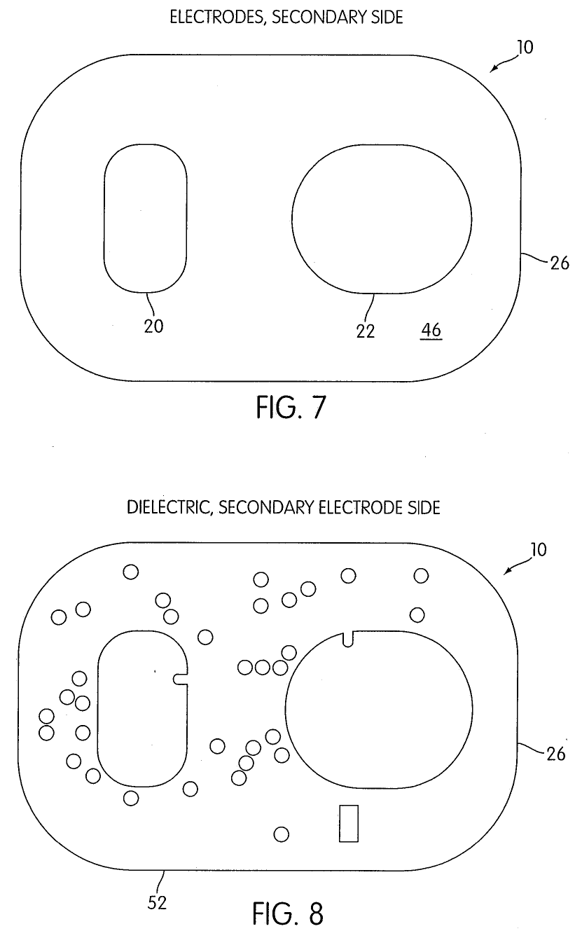

FIG. 7 depicts a formation of electrodes 20 and 22 on secondary component side 46 of layer 26. Electrodes 20 and 22 are formed on top of wirings 48. Electrodes 20 and 22 are formed of silver or silver chloride. In a preferred embodiment, wirings 48 have a higher resistivity than electrodes 20 and 22. Electrodes 20 and 22 may be made from a material having a resistivity lower than wirings 48 in order to deliver a desirable amount of electricity to a patient's skin that is just below a patient's sensory perception. Thus, in addition to varying electrode size to alter the amount of electricity delivered by electrodes 20 and 22 to accommodate drug molecules of varying sizes, the materials used to form electrodes 20 and 22 may also be varied to affect these parameters as well.

The larger of the two electrodes 22 would contain the positively or negatively charged drug molecule. The smaller of the two electrodes 20 would be the return and would contain only the hydrogel material. For positively charged drug molecules, the larger electrode 22 is constructed of silver ink with one or multiple print passes as well as varied silver loading. The return electrode 20 is constructed of silver/silver chloride ink with one or multiple print passes as well as varied silver chloride loading. For a negatively charged drug molecules, the larger electrode 22 is constructed of silver/silver chloride ink with one or multiple print passes as well as varied silver chloride loading. The return electrode 20 is constructed of silver ink with one or multiple print passes as well as varied silver loading.

This combination of material and material sets enhances the drug delivery performance, stabilizes the pH and increases the delivery time of the patch system.

FIG. 8 depicts a deposition of dielectric material 52 on secondary component side 46 of layer 26. Dielectric material 52 is deposited to cover wirings 50. The dielectric material is not deposited on electrodes 20 or 22.

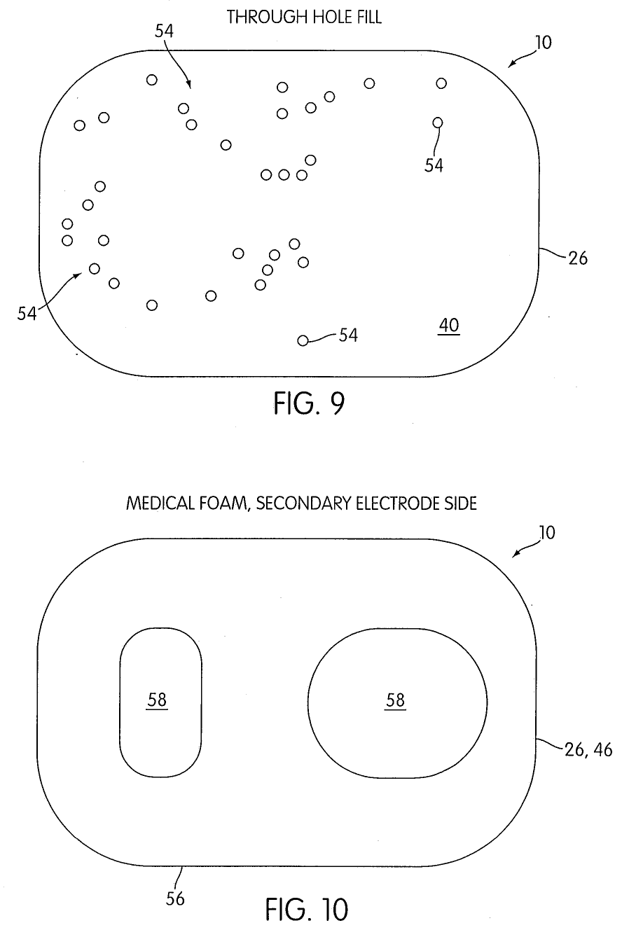

FIG. 9 depicts a filing of through holes 54 in layer 26. Through holes 54 are filled with a conductive material in order to electrically couple wirings 50 to circuitry 38. This conductive material is preferably printed silver ink.

FIG. 10 depicts the attachment of laser or die cut foam 56 to secondary component side 46 of layer 26. Foam 56 is cut to have openings 58. Openings 58 are provided for the formation of drug reservoirs 24. Openings 58 coincide with the position of electrodes 20 and 22 on top of which drug reservoirs 24 are formed. Foam 56 is attached to secondary component side 46 of layer 26. In another embodiment, printed silicone adhesive is used in place of foam 56.

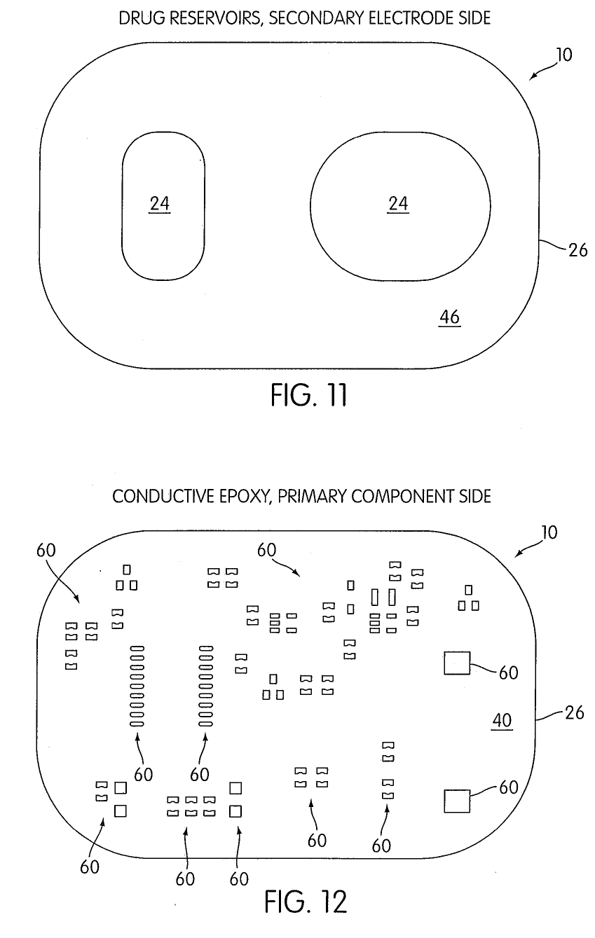

FIG. 11 depicts a formation of drug reservoirs 24 on secondary component side 46 of layer 26. In this exemplary embodiment, drug reservoirs 24 are formed from hydro-gel that is deposited within openings 58 of foam 56 over electrodes 20 and 22.

FIG. 12 depicts a deposition of conductive epoxy 60 on primary component side 40 of layer 26. Conductive epoxy 60 is deposited in the pattern shown in FIG. 12 to secure microprocessor controller 12 and battery 14 onto layer 26 and place those components into electrical connection with circuitry 38.

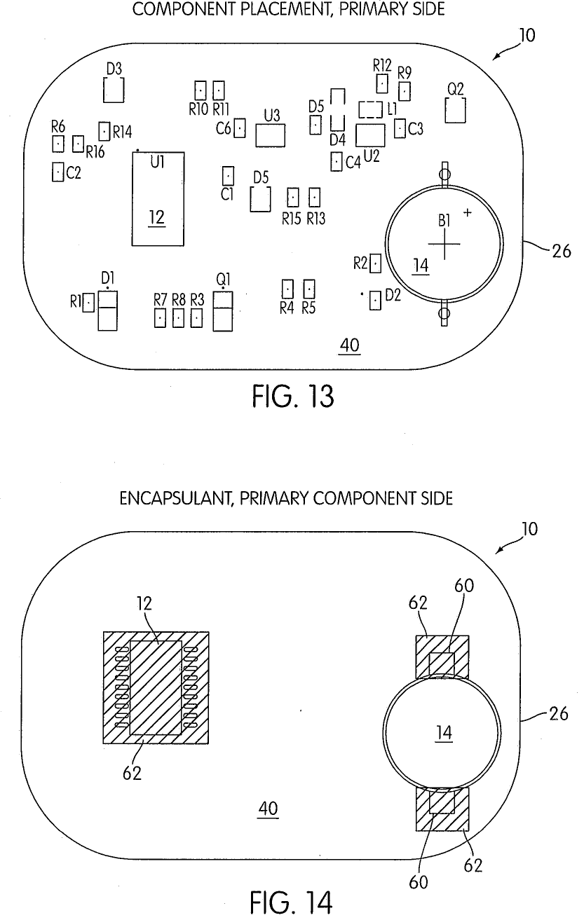

FIG. 13 depicts a placement of components 12 and 14 on primary component side 40 of layer 26. Microprocessor 12 and battery 14 are attached to layer 26 over the positions where conductive epoxy 60 (shown in FIG. 12) was deposited. The components labeled with the label “D” are diodes, the components labeled with “C” are capacitors, and the components labeled with “R” are resistors.

FIG. 14 depicts a deposition of an encapsulant material 62 on primary component side 40 of layer 26. Encapsulant material 62 covers the electrical connections that microprocessor 12 and battery 14 form with circuitry 38. Encapsulant material 62 is used to protect the electrical connections that microprocessor 12 and battery 14 form with circuitry 38 from damage from moisture or other contaminants. Encapsulant material 62, in one exemplary embodiment, is a Ultra-Violet (UV) curable encapsulation photopolymer designed to secure low profile surface mount devices to a flexible substrate.

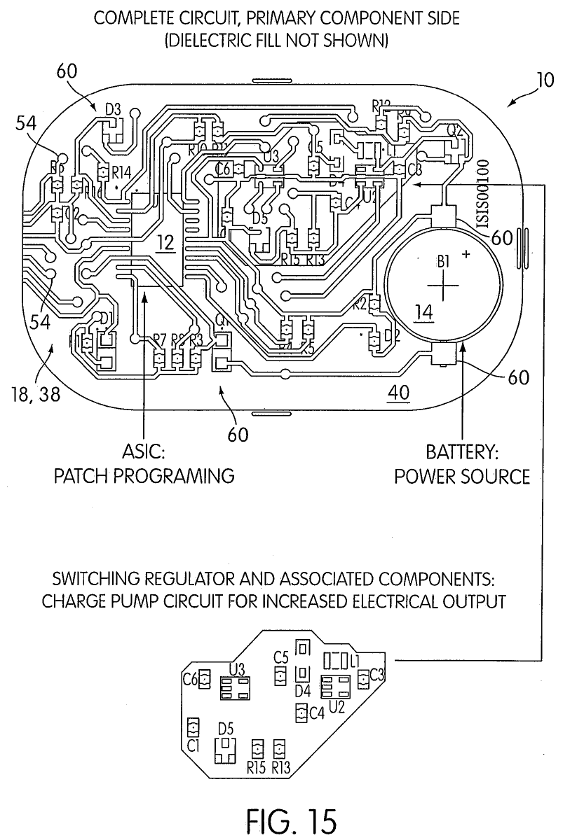

FIG. 15 illustrates a completed primary component side 40 of layer 26. Microprocessor controller 12 and battery are mounted to layer 26. Antenna 16 is formed and connected to microprocessor controller 12 with wirings 18. Through holes 54 interconnect microcontroller 12 and battery 14 to electrodes 20 and 22 on the secondary component side 46 of layer 26. Circuitry 38 includes a switching regulator and associated components as well as a charge pump for increased electrical output.

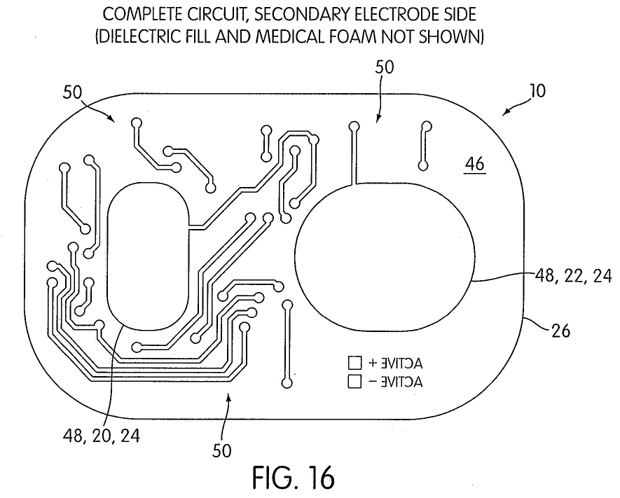

FIG. 16 illustrates a completed secondary component side 46 of layer 26. Drug reservoirs 24 are formed over electrodes 20 and 22 and are surrounded by foam tape 56. The outer edges of secondary component side 46 are covered with high-tack adhesive 30. The central portion of secondary component side 46 is covered with low-tack adhesive. Wirings 50 connect electrodes 20 and 22 to battery 14 and microprocessor controller 12 by through holes 54.

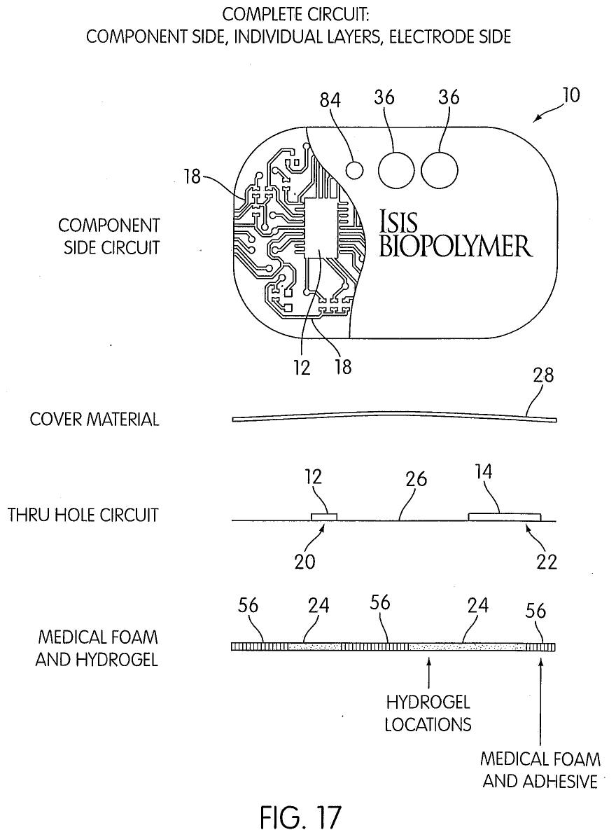

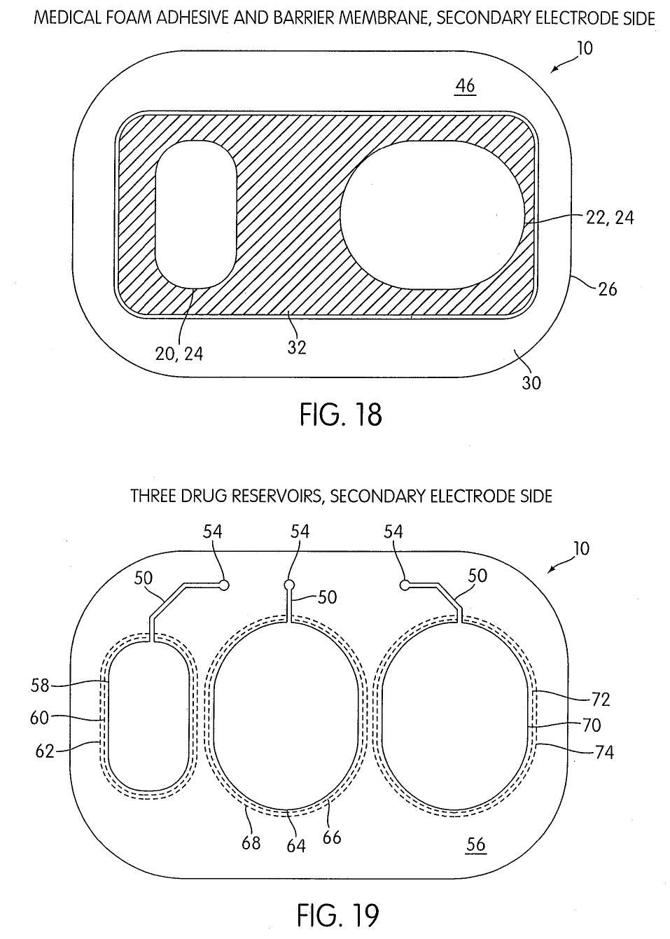

FIG. 17 illustrates a side view of iontophoretic drug delivery system 10. Layer 28 is shown covering microprocessor controller 12, battery 14, and antenna 16. Microprocessor controller 12, battery 14 and antenna 16 are attached to primary component side 40 of layer 26. On the secondary component side 46 of layer 26, electrodes 20 and 22 are printed on layer 26. Layer 26 is attached to foam layer 56, in which drug chambers 24 are formed. Adhesives 30 and 32 are placed on the bottom surface of layer 56 (as shown in FIG. 18).

FIG. 18 illustrates an adhesive pattern on secondary component side 46 of layer 26. The peripheral portion of secondary component side 46 is covered with high tack adhesive 30. The dashed inner portion of secondary component side 46 is covered with low tack adhesive 32. Electrodes 20 and 22 and drug chambers 24 are not covered with any adhesive so that the adhesive does not interfere with the transference of charged drug molecules from drug chambers 24 into the patient's skin.

FIG. 19 illustrates an alternative embodiment for iontophoretic drug delivery system 10. System 10 includes a first drug reservoir 58 formed on an electrode 60, which is formed on printed circuit 62. System 10 includes a second drug reservoir 64 formed on an electrode 66, which is formed on printed circuit 68. System 10 also includes a third drug reservoir 70 formed on electrode 72, which is formed on printed circuit 74. Printed circuits 62, 68 and 74 are connected with printed wirings 50 that lead to through holes 54. Electrodes 60, 66, and 70 are coupled to separate terminals of microprocessor controller 12 and are operated independently of each other by microprocessor controller 12. Electrodes 60, 66 and 70 are varied in size according to the variance in size of the charged drug molecules that electrodes 60, 66 and 70 drive into a patient's skin.

FIG. 20 illustrates a side view of a manual button array 36 for manually operating an iontophoretic drug delivery system 10. Manual button array, in this exemplary non-limiting embodiment, is formed of one or more poly-dome switch assemblies 36. Poly-dome switch assemblies 36.

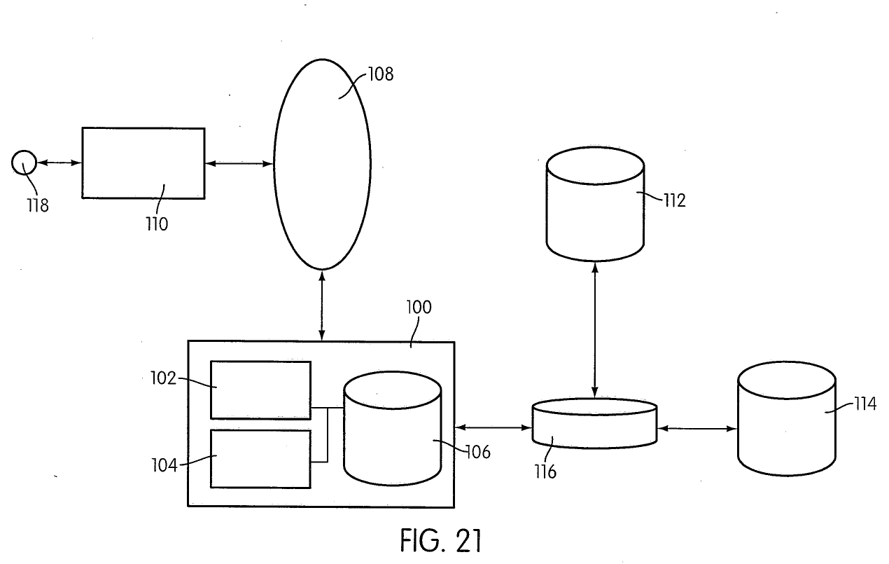

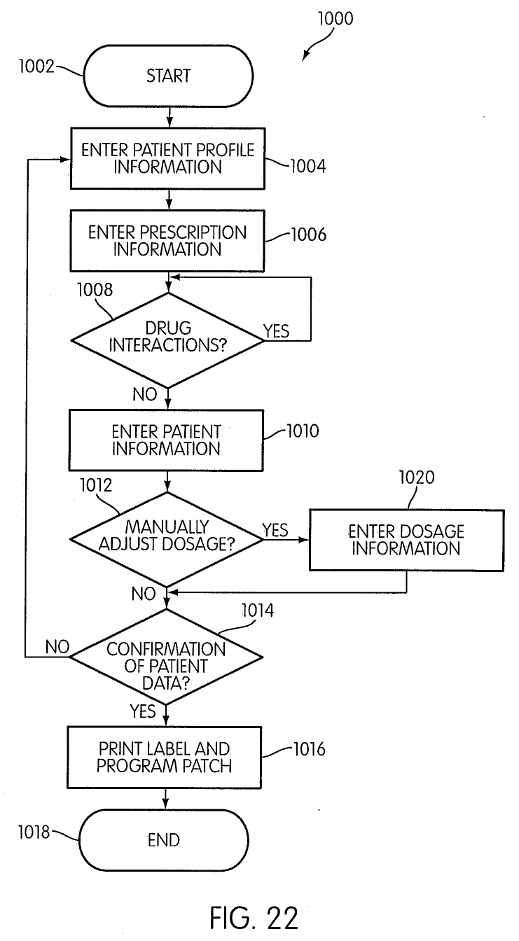

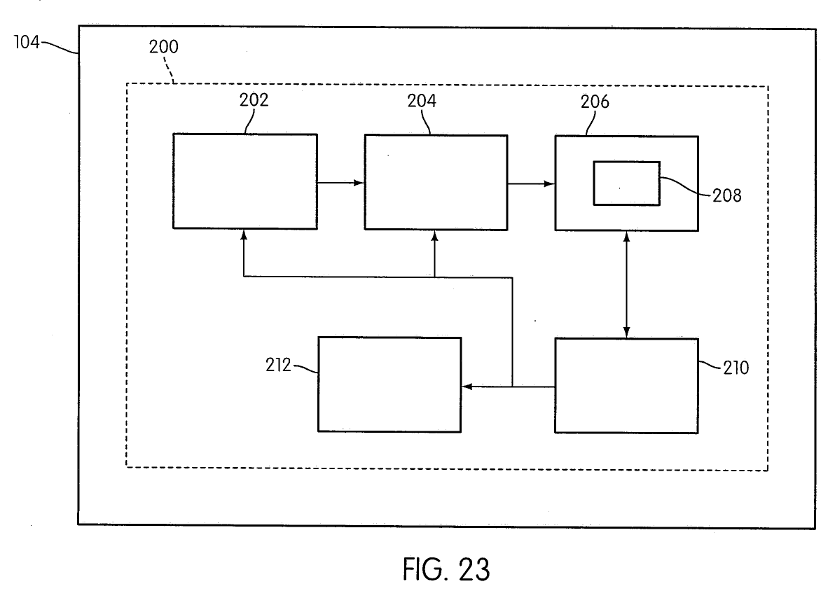

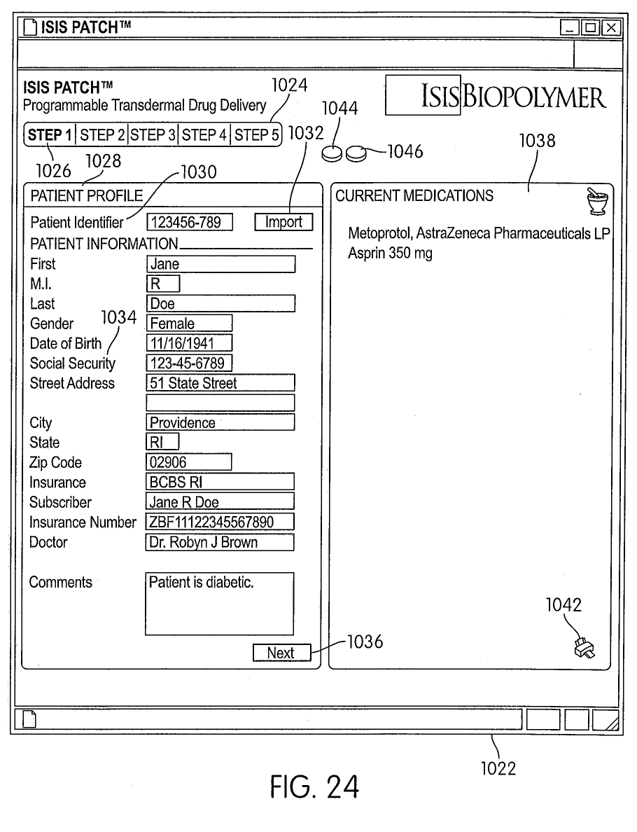

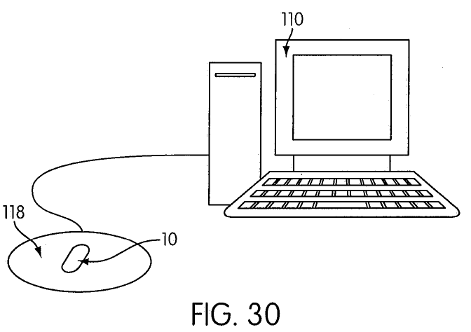

Iontophoretic drug delivery system 10 maybe prescribed and programmed through the use of a computer network system and associated software. FIG. 21 illustrates a block diagram of an exemplary network system for prescribing medication and programming an iontophoretic drug delivery system 10. FIG. 22 illustrates a flow chart depicting an exemplary software process for prescribing medication and programming an iontophoretic drug delivery system 10. FIG. 23 illustrates a software module diagram of the software for prescribing medication and programming an iontophoretic drug delivery system 10. FIGS. 24-29 illustrate screen shots of the software program for prescribing and programming an iontophoretic drug delivery system 10. FIG. 30 illustrates a computer terminal equipped with a wireless device for programming the iontophoretic drug delivery system 10.

Referring to FIG. 21, a computer support system 100 is shown. Computer support system 100 includes a web server 102, an application server 104, and a database 106. Computer support system 100 connects through a network, such as the Internet 108, to at least one computer terminal 110. Computer support system 100 may also connect to databases 112 and 114 through an SQL server agent 116. Computer support system 100 supports a software application for prescribing and programming iontophoretic drug delivery system 10.

Computer terminal 110, in a preferred embodiment, is a computer terminal located in a pharmacy. A pharmacist seeking to fill a prescription for iontophoretic drug delivery system 10 would first access computer terminal 110. Computer terminal 110 can access the application for prescribing and programming the iontophoretic drug delivery system 10 supported on computer support system 100 through Internet 108. While FIG. 21 illustrates a single computer terminal 110, it is envisioned that a multitude of computer terminals 110 would interface with computer support system 100 through Internet 108. This multitude of computer terminals 110 would, for example, be located at pharmacies throughout a geographic area. Computer terminal 110, in an exemplary embodiment, is a conventional computer equipped with an operating system, a graphical user interface, and a web browser configured to communicate with computer support system 100 through Internet 108.

Web server 102 is a computer that supports software responsible for receiving requests from and sending responses to the web browser supported by computer terminal 110. These responses can include web pages and other linked content. Preferably these requests and responses are based upon the application software described in FIGS. 22-29. Web server 102 is in communication with application server 104. Application server 104 supports the software for prescribing and programming the iontophoretic drug delivery system 10. Web server 102 and application server 104 communicate with database 106. Database 106 stores information related to the software for prescribing and programming the iontophoretic drug delivery system 10, such as pharmaceutical information, patient information, device programming information, pharmacy information, and other related information. Exemplary pharmaceutical information can include drug interaction information to enable the prevention of interactions with other prescribed medication. Other exemplary pharmaceutical information can include dosage schedules and serum concentration information based upon patient parameters such as gender, weight, height, and age. Further pharmaceutical information can include target blood saturation levels and prescription periods. Exemplary patient information can include the patient's name, social security number, date of birth, address, telephone number, insurance provider information, doctor information, information related to prescriptions such as allergies, and other patient medical information. Device programming information can include the information related to programming the iontophoretic drug delivery system 10 such as the dosage cycle, prescription concentration level, information related to the time periods for turning the electrodes ON and OFF, information related to the voltage placed across the electrodes, and other information for programming system 10. Pharmacy information can include business information related to the particular pharmacy, such as pharmacy locations, sales information concerning iontophoretic drug delivery system 10, stocking information related to iontophoretic drug delivery system 10, and other related business information.

Computer support system 100 communicates through SQL server agent 116 with databases 112 and 114. Database 112, in this exemplary embodiment, is a database supported by a pharmacy. Database 112 may contain information related to the prescribing of medication through the iontophoretic drug delivery system 10 and the particular patient. Database 114, in this exemplary embodiment, is a database supported by a pharmaceutical company. Database 114 may contain pharmaceutical information and information related to the medications delivered by the iontophoretic drug delivery system 10. Databases 1 12 and 114 function to provide additional information to system 100 as needed to support the prescription of the iontophoretic drug delivery system 10.

Once a pharmacist has completed the prescription process with the software application to create a prescription, the software application generates programming instructions for iontophoretic drug delivery system 10. Computer support system 100 transmits these instructions via Internet 108 to computer terminal 110. A programming device 118 is connected to computer terminal 110. The programming device 118 is preferably wireless, but may also connect to the iontophoretic drug delivery system 10 by a wired connection, such as a USB connection. Wireless programming device 118 is configured to transmit programming instructions to iontophoretic drug delivery system 10 from computer terminal 110 on how to function, operate, and deliver the medication to the patient. Once programmed with these instructions, the iontophoretic drug delivery system 10 is ready to be dispensed to a patient.

FIG. 22 illustrates a flow chart 1000 depicting a process for prescribing and programming the iontophoretic drug delivery system 10 using the application software supported by computer support system 100. A pharmacist starts the process in step 1002 by accessing a Internet capable computer terminal 110 and utilizing the web browser to access the application software supported on computer support system 100. Once the pharmacist has accessed the application software, the pharmacist will enter patient profile information in step 1004. Then the pharmacist will enter prescription information in step 1006. In step 1008, the software application will access databases 106, 112 and 114 to ascertain if there are any potential drug interactions with the patient's current prescriptions and the prescription for the iontophoretic drug delivery system 10. The application software will not permit the prescription process 1000 to continue until all drug interactions have been resolved. Once all drug interactions have been resolved, the process 1000 proceeds to step 1010 where patient information is entered. Patient information includes physical characteristics of the particular patient to facilitate the proper prescription, such as race, renal function, diet, level of lifestyle activity (exercise, sports, etc.), and amount of sleep per day.

Databases 106, 112, and 114 store default programming information including dosage cycles and concentrations for particular medications and specific patient profiles. In step 1012, the pharmacist can elect whether to accept this default dosage program, or elect to manually adjust the dosage information in step 1020. In step 1020, the pharmacist may specifically tailor the dosage schedule and dosage concentration level for a particular patient. For example, the dosage schedule may be tailored to accommodate the particular patient's eating and sleeping schedules, or the dosage schedule may be ordered by the prescribing doctor for medical reasons.

In step 1014, the pharmacist has an opportunity to review all of the patient data and prescription information. If any of that information is incorrect, the pharmacist can return to steps 1010, 1012 and 1020 and revise any of that information. Once all of the information is correct, the pharmacist can proceed to step 1016 where the prescription label is prepared and the patch is programmed by computer terminal 110 with wireless programming device 118. The process terminates with step 1018 where the iontophoretic drug delivery system 10 is programmed and ready to be provided to a patient with the appropriate label.

FIG. 23 illustrates a software module diagram 200 of the software application for prescribing medication and programming an iontophoretic drug delivery system 10. The software application is supported in application server 104 on computer support system 100. The software application includes a patient profile software module 202, a prescription information software module 204, a patient information software module 206, a manual level adjustment module 208, a confirmation module 210, and a prescription module 212.

The patient profile software module 202 is configured to acquire various patient information on the patient through the web browser supported by computer terminal 110. The patient profile software module 202 performs step 1004 in FIG. 22. This patient information can include the patient's name, social security number, date of birth, address, telephone number, insurance provider information, doctor information, information related to prescriptions such as allergies, and other patient medical information. The patient profile software module 202 gathers this patient information through the web browser supported on computer terminal 110 and stores it in database 106.

The prescription information software module 204 is configured to gather drug information through the web browser supported by computer terminal 110. This drug information can include the commercial name of a drug, the name of the chemical compound for the drug, the dosage amount, the dosage frequency, the manufacturer of the drug, and the drug regimen. Other drug information can include a description of the target blood saturation level, the duration of the drug treatment, and patient details such as gender, height, weight, age, and race. The prescription information software module 204 gathers the above information and accesses databases 106, 112, and 114 to ascertain whether the specific patient identified by the patient profile software module 202 has any other existing prescriptions and whether or not those prescriptions would interact with the current prescription. In the event there is an interaction, the prescription information software module 204 creates a warning message that is sent for display on the web browser supported on computer terminal 110. The prescription information software module 204 will prevent further progress in the prescription process until all drug interactions have been resolved. The prescription information software module 204 performs steps 1006 and 1008 in FIG. 22.

The patient information software module 206 is configured to gather patient information directly related to the prescribed medication. This patient information can include the patient's race, renal function, diet, number of hours spent sleeping daily, as well as their level of daily activity. Utilizing this information, the patient information software module 206 accesses databases 106, 112, and 114 to acquire a default dosage program for the particular patient for the prescribed medication. The user can decide whether they wish to prescribe this default dosage program, or manually create a different dosage program using manual level adjustment module 208. With manual level adjustment module 208, the user can manually set the dosage schedule to custom tailor it to accommodate for meals, sleeping periods, and other activities. The user can also set the various dosage concentration levels in response to the patient's particular daily lifestyle. The manual level adjustment module 208 is a sub-module within the patient information software module 206. When a user, such as a pharmacist, wants to manually adjust the default dosage profile selected by the patient information software module 206, the patient information software module 206 accesses the manual level adjustment module 208. Once the user has completed use of manual level adjustment module 208, manual level adjustment module 208 returns the user to the patient information software module 206. The patient information software module 206 performs step 1010 and 1012 in FIG. 22. The manual level adjustment module 208 performs step 1020 in FIG. 22.

The confirmation module 210 presents the user with the opportunity to confirm the information entered into the patient profile software module 202, prescription information software module 204, patient information software module 206, and manual level adjustment module 208. In the event that any of the information is inaccurate, the user has the opportunity to return to the previous software modules 202, 204, 206 and 208 to correct the information. Once the user has confirmed all of the information is accurate, which is shown as step 1014 in FIG. 22, the confirmation module hands the user off to the prescription module 212.

The prescription module 212 is configured to print a prescription label. The prescription label can include patient information such as the patient's name and address. The prescription label can also include pharmaceutical information such as the name of the drug contained in the iontophoretic drug delivery system 10, possible side effects of the prescribed drug, various instructions to the patient regarding the drug or the use of the patch, the name of the prescribing physician, and a bar code to identify the specific prescription. The prescription label, created by computer support system 100, is then printed out on a printer attached to computer terminal 110 by means of the supported web browser.

The prescription module 212 is also configured to create and transmit the programming instructions for the iontophoretic drug delivery system 10. The user instructs the prescription module 212 to create and transmit these programming instructions to the iontophoretic drug delivery system 10. The programming instructions are based upon information entered into the prescription module 206 and the manual level adjustment module 208 and information stored in databases 106, 112 and 114. The computer support system 100 then transmits the programming instructions across Internet 108 to computer terminal 110 where they are received by the supported browser. Computer terminal 110 then sends these commands to the iontophoretic drug delivery system 10 through the wireless programming device 118. The steps of printing the label and programming the iontophoretic drug delivery system 10 are shown as step 1016 in FIG. 22. The microprocessor controller 12 is pre-programmed during the manufacturing process to include base firmware. During the programming sequence outlined in step 1016 of FIG. 22, the iontophoretic drug delivery system 10 will just receive the parameters that were established specific to that patient. The base firmware contains safe-guards so that the pharmacist or doctor cannot prescribe more than the recommended dosage amount or a target serum level that exceeds the recommended limit

FIG. 24 illustrates a screen shot 1022 of a patient profile software module 202. Screen shot 1022 is sent by web server 102 over Internet 108 to computer terminal 110 where it is displayed using the supported web browser. Screen shot 1022 includes a progress bar 1024 at the top of the screen 1022. Progress bar 1024 lists the five primary steps in the prescription process, listed as step 1, step 2, step 3, step 4, and step 5. These five primary steps correspond to steps 1004, 1006, 1010, 1014 and 1016 shown in FIG. 22. The progress bar 1024 includes a highlighted step identifier 1026 to indicate the current step. In screen shot 1022, progress bar 1024 has step 1 signified by highlighted step identifier 1026, showing that screen shot 1022 is at step 1004 shown in FIG. 22. Corresponding to step 1004 in FIG. 22, screen shot 1022 shows the patient profile screen 1028 supported by patient profile module 202.

A user may enter the patient's identifier number in section 1030. With this identifier number 1030, the user may use button 1032 to import the patient's information stored in a database 106, 112, or 114 to complete the patient information form 1034. The user may edit the information in form 1034 to ensure that it is current. This patient information can include the patient's name, social security number, date of birth, street address, insurer information, doctor information, and other comments. After completing form 1034, the user may select button 1036 to advance to the next screen. Section 1038 provides a listing of all current medications that the patient identified by patient identifier number 1030. The user can email information from this page using an optional GUI button (not shown), or choose to print screen shot 1022 using button 1042. Button 1044 is an information button. Button 1044 may provide information regarding the software application, regarding the company supporting the software application, or other information related to the software or the prescription process. That information may include contact information. Button 1046 provides a link to where users can seek answers for questions. Button 1046 may link to a page listing frequently asked questions and answers. Alternatively, button 1046 may provide a link to a live chat session with an online help person. Button 1046 may also provide a listing of contact information where the user may seek answers for their questions.

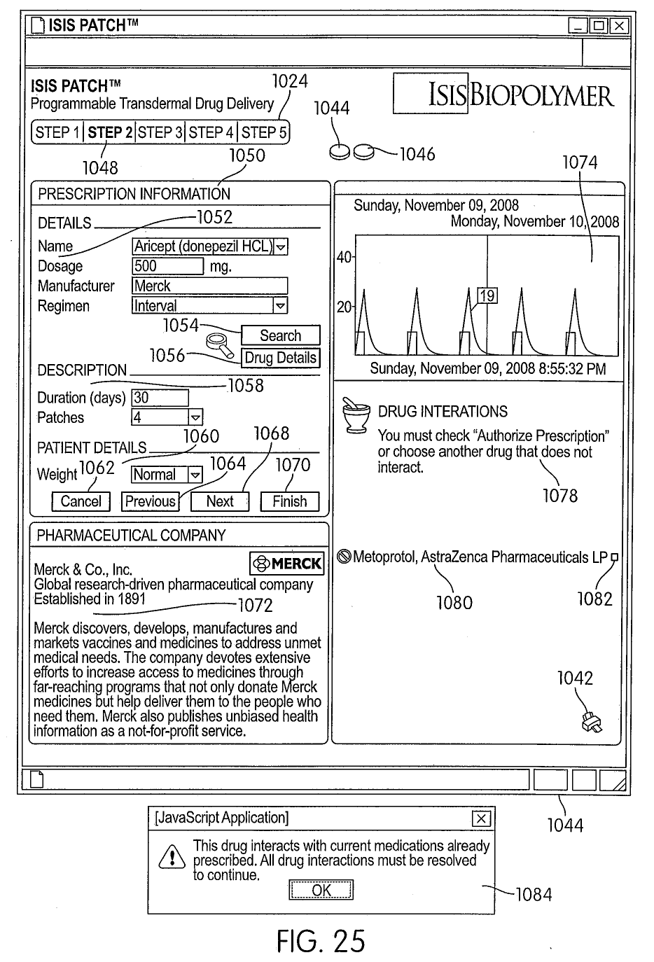

FIG. 25 illustrates a screen shot 1044 of a prescription information software module 204. Progress bar 1024 shows that step 2 is identified as the current step by the highlighted step identifier 1048. The prescription information screen 1050 includes a section for drug information 1052. This information can include the name of the drug, the dosage amount, the dosage frequency, the name of the manufacturer, and the prescribed regimen. Using button 1054, a user may enter the name of the drug and search for other drug information based upon the name for completing form 1050. A user may search for additional details on the drug using button 1056. Section 1058 includes a description of the drug prescription, including the target blood saturation level, the duration of the treatment, and the number of iontophoretic drug delivery systems 4, i.e. “patches,” to be prescribed. In this example, four patches 1058 are prescribed. In section 1060, the user may enter patient details pertinent to the prescription dosage such as gender, weight, height, and age.

Once form 1050 is completed, the user may use button 1062 to cancel the order, button 1064 to return to a previous screen, button 1068 to move to the next screen, or button 1070 to finalize the information entered and proceed to the next screen. Section 1072 provides a description of the manufacturer of the medication listed in section 1052.

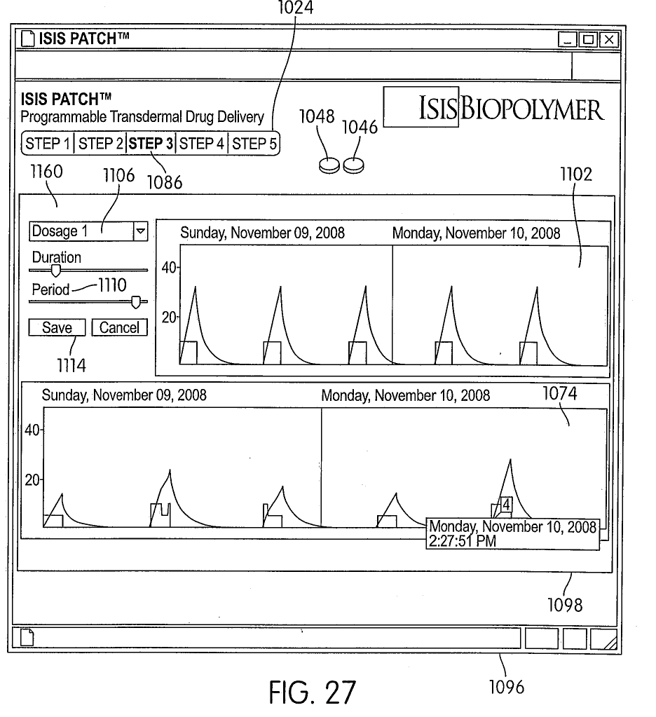

Based upon the information listed in section 1050, the prescription information module 204 accesses a default dosage schedule shown in FIG. 1074, which is a default prescription profile. FIG. 1074 is acquired from one of databases 106, 112, or 114. FIG. 1074 shows the dosage level as a function of time. In this case, the dosage frequency has the form of a square wave. Based upon this dosage schedule, FIG. 1074 shows the projected serum concentration in the patient as a function of time. Utilizing button 1068, the user may move to the next screen and deviate from this default dosage schedule and manually select the dosage schedule. The manual selection of the dosage schedule is shown in detail in FIG. 27.

Section 1078 provides a listing of any drug interactions 1080 that may occur with any of the patients existing medications. A check box 1082 is provided for each of the drug interactions. Warning screen 1084 is displayed when the user fails to resolve all of the drug interactions. The user must press okay on screen 1084 to return to screen show 1044 in order to resolve all drug interactions. Completed steps are shown completed in the progress bar with darkened identifiers 1084.

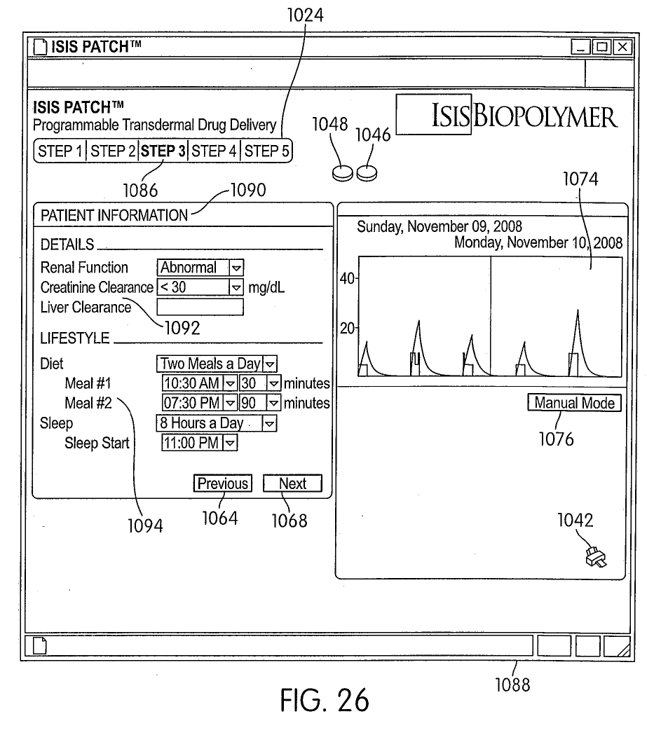

FIG. 26 illustrates a screen shot 1088 of a patient information software module 206 having a default prescription profile 1074. Progress bar 1024 shows that the user is currently on step 3 via identifier 1086. In section 1090, the user can enter patient information pertinent to the prescription. This information can include details related to the patient's renal function, or creatinine clearance level as shown in section 1092. Further, the user may enter information related to the patient's lifestyle in section 1094, such as their diet, lifestyle, and sleeping schedule.

FIG. 27 illustrates a screen shot 1096 of a manual level adjustment module 208. Screen shot 1096 and manual adjustment module 208 is reached by selecting button 1068. Screen shot 1096 displays the default dosage FIG. 1074 in section 1098 and provides a manual adjustment section 1100. In section 1100, the user may manually alter the default dosage schedule. To create a custom dosage schedule 1102, the user may manually adjust the regimen by selecting a particular dosage 1106, or set dosage levels to zero. Utilizing tool 1110, the user may specify the dosage amount and dosage duration for a particular dosage 1106. Utilizing the manual adjustment features 1106 and 1110, the user may create a custom dosage schedule 1102. The default dosage schedule 1074 shows the dosage amount as a frequency of time consisting of a continuous square wave. In comparison, the custom manually set dosage schedule 1102 includes deviations in the default square wave dosage schedule to accommodate for meals, such as breakfast, lunch and dinner, as well as the patient's sleep period. Thus, with the manual adjustment feature, the user can create a dosage schedule specifically tailored for the particular patient. Once the custom manually set dosage profile 1102 is finalized, the user can save the custom manually set dosage profile and return to screen shot 1088 by selecting the finalize button 1114. The profile of the dosage schedule shown in 1102 as a modified square wave corresponds to the operation of device 10. When the modified square wave has zero amplitude, the electrodes 20 and 22 are turned OFF. When the modified square wave has a non-zero amplitude, the electrodes 20 and 22 are turned ON and are charged with a level of voltage corresponding to the varying concentration level during the dosage schedule. The time varying dosage information therefore corresponds to ON duration period information for the electrodes 20 and 22. The concentration level dosage information corresponds to voltage level information for the electrodes 20 and 22.

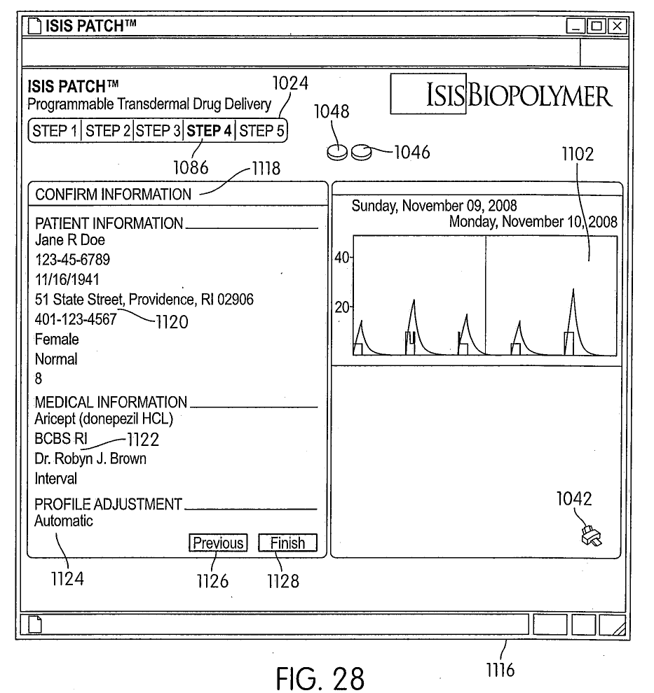

FIG. 28 illustrates a screen shot 1116 of a confirmation software module 212. The progress bar 1024 illustrates that step 4 is highlighted 1086, which corresponds to step 1014 in FIG. 22. Steps 1, 2 and 3, having been completed, are darkened with identifiers 1084. Section 1118 lists a confirmation of the information provided in steps 1, 2 and 3. This information includes patient information listed in section 1020, medication information in section 1122, an indicator as to whether a custom or manual dosage profile is being used, and a display of FIG. 1102 showing the prescription.

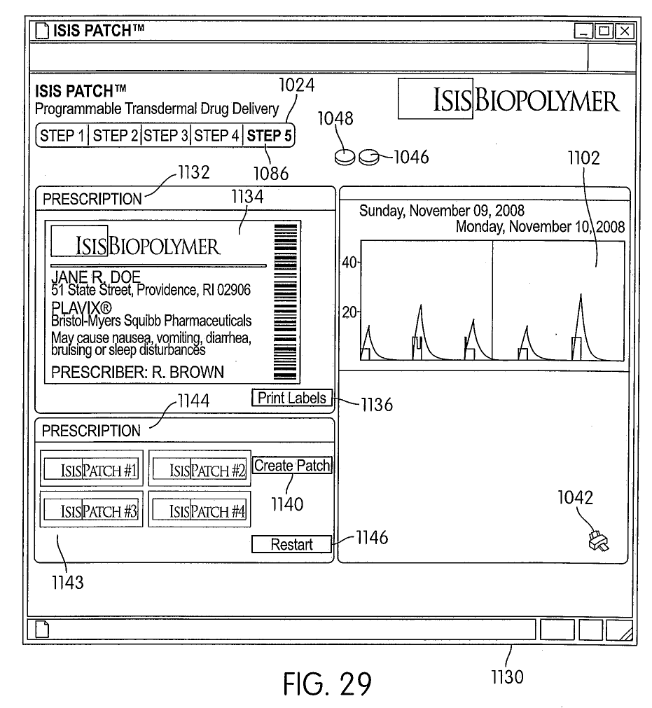

FIG. 29 illustrates a screen shot 1130 of a prescription module 212 for printing prescription labels 1034. Screen shot 1030 illustrates that the user has reached step 5 in the progress bar 1024 with highlighted identifier 1086. Step 5 corresponds to step 1016 in FIG. 22. In section 1032, the prescription label 1034 is shown. The prescription label 1034 includes the name and address of the patient, the name of the drug and its manufacturer, various warnings and other instructional information. The user can print the label 1034 from computer terminal 110 using button 1036 and an attached printer. The user can select button 1140 to create the programming instructions for the iontophoretic patch based on the dosage schedule shown in FIG. 1102. Once a user has completed the prescription process illustrated in FIG. 22 and steps 1-5 in FIGS. 24-32, the final step is executed by selecting button 1140 to create iontophoretic drug delivery system 10. Selecting button 1140 causes application server 104 to access database 106 to produce programming instructions for iontophoretic drug delivery system 10 based upon FIG. 1102. Those instructions are transmitted by web server 102 across internet 108 where they are received by the web browser supported on computer terminal 110. Computer terminal 110 then uses wireless programming device 118 to wirelessly transmit those programming instructions to iontophoretic drug delivery system 10. In this example, as noted in FIG. 25, the pharmacist selected the creation of four iontophoretic drug delivery systems 10. Section 1143 illustrates four separate icons, each of which symbolizes one of the four patches 10 prescribed in FIG. 25. The use of four icons is merely exemplary. The number of icons in section 1143 will correspond to the number of patches 10 prescribed in section 1058 of FIG. 25. The upper left icon identifying patch number 1 in section 1143 has a check mark over it signifying that the patch is complete along with text below the icon stating that the patch is complete. The upper right icon identifying patch number 2 in section 1143 has text below stating that patch number 2 is in the process of being created. Progress indicator 1144 illustrates the processing and progress of this creation and transmission of the programming instructions to patch number 2.

FIG. 30 illustrates an isometric view of a iontophoretic drug delivery system 10 being wirelessly programmed. Computer terminal 110 is connected to a wireless programming device 118. As discussed earlier, wireless programming device 118 transmits the programming instructions generated by computer support system 100 to the iontophoretic drug delivery device 10. Wireless programming device 118 may transmit these programming instructions to antenna 16 on device 10 with electromagnetic signals, capacitive coupling, inductive coupling, infra-red signaling, or another wireless manner. Iontophoretic drug delivery device 10 is shown resting on programming device 118 in this exemplary embodiment while it is being programmed.

While the invention has been shown and described with reference to a particular embodiment thereof, it will be understood to those skilled in the art, that various changes in form and details may be made therein without departing from the spirit and scope of the invention.

What is claimed is:

1. An iontophoretic drug delivery system for driving charged drug molecules into a tissue, comprising:
a flexible patch, comprising:
a drug reservoir holding the charged drug molecules;
an electrode for driving the charged drug molecules into the tissue;
a microprocessor controller configured to control the electrode;
a receiver coupled to the microprocessor controller for enabling the microprocessor controller to be programmed by machine readable programming instructions sent through the receiver; and
a battery coupled to the microprocessor controller;
a software module for creating a set of machine readable programming instructions for the microprocessor to control the operation of the electrode to administer the charged drug molecules held in the drug reservoir; and
a programming device for communicating the machine readable programming instructions to the microprocessor through the receiver.

2. The system of claim 1, wherein the machine readable programming instructions include duration information for turning the electrode ON and OFF.

3. The system of claim 2, wherein the machine readable programming instructions include voltage information for a level of voltage placed across the electrode when turned ON.

4. The system of claim 3, wherein the machine readable programming instructions are selected from a database based upon a type of the charged drug molecules and a patient parameter.

5. The system of claim 3, wherein the machine readable programming instructions are created manually by a user using the software module.

6. The system of claim 1, wherein the microprocessor controller is connected to the electrodes with flexible printed wires.

7. The system of claim 6, wherein the flexible printed wires are made of silver or silver chloride.

8. The system of claim 6, wherein said microprocessor controller and electrodes are connected to the flexible printed wires with a conductive cement.

9. The system of claim 1, further comprising a high-tack adhesive placed around an outer edge of the iontophoretic drug delivery system and a low-tack adhesive placed in a center of the iontophoretic drug delivery system.

10. The system of claim 1, further comprising a tissue permeation enhancer configured to enhance permeation of the drug molecule into the tissue.

11. The system of claim 10, wherein the permeation enhancer is in the reservoir.

12. The system of claim 10, wherein the permeation enhancer is an excipient.

13. The system of claim 1, wherein the programming device connects to the receiver with a wireless connection.

14. The system of claim 1, wherein the programming device connects to the receiver with a wired connection.

15. The system of claim 14, wherein the wired connection is a USB connection.

16. The system of claim 1, further comprising a sensor connected to the microprocessor controller, the sensor being configured to monitor an environmental condition and provide a feedback to the microprocessor controller.

17. The system of claim 16, wherein the environmental condition is selected from the group of environmental conditions consisting of: moisture, temperature, physical contact between a patient and the iontophoretic drug delivery device, skin temperature, and heart rate.

18. The system of claim 17, wherein in response to the feedback provided by the sensor, the microprocessor control is configured to perform an action selected from the group of actions consisting of: turning the iontophoretic drug delivery device ON, turning the iontophoretic drug delivery device OFF, and varying a voltage of the electrode.

19. A method of programming an iontophoretic drug delivery device, comprising:
obtaining a set of machine readable iontophoretic programming instructions based on prescription information and user-specific information; and
transmitting the machine readable iontophoretic programming instructions from the system to the iontophoretic drug delivery device.

20. The method of claim 19, wherein the machine readable iontophoretic programming instructions comprise dosage cycle information.

21. The method of claim 19, wherein the machine readable iontophoretic programming instructions comprise prescription concentration level information.

22. The method of claim 19, wherein the iontophoretic drug delivery device comprises an electrode, wherein the machine readable iontophoretic programming instructions comprise time information for regulating time periods during which the electrode is ON and OFF.

23. The method of claim 19, wherein the iontophoretic drug delivery device comprises an electrode, wherein the machine readable iontophoretic programming instructions comprise voltage information for regulating a voltage of the electrode.

24. The method of claim 19, further comprising determining whether a drug interaction can occur from the prescription information.

25. The method of claim 19, further comprising manually creating a set of machine readable iontophoretic programming instructions with a Graphical User Interface.

26. The method of claim 25, wherein manually creating a set of machine readable iontophoretic programming instructions with a Graphical User Interface comprises:
selecting a particular dosage;
selecting a duration for the dosage; and
selecting a dosage period.

27. The method of claim 19, further comprising:
entering patient information into the system; and
forming a label containing at least a portion of the patient information and the prescription information.

28. The method of claim 19, wherein the machine readable iontophoretic programming instructions are transmitted from the system to the iontophoretic drug delivery device wirelessly.

29. The method of claim 19, wherein the machine readable iontophoretic programming instructions are transmitted from the system to the iontophoretic drug delivery device through a wired connection.

30. The method of claim 29, wherein the wired connection is a USB connection.

31. An iontophoretic drug delivery system for driving charged drug molecules into a tissue, comprising:
a flexible patch, comprising:
a drug reservoir holding the charged drug molecules;
an electrode for driving the charged drug molecules into the tissue;
a microprocessor controller configured to control the electrode;
a receiver coupled to the microprocessor controller for enabling the microprocessor controller to be programmed by machine readable programming instructions sent through the receiver; and
a battery coupled to the microprocessor controller; and
a programming device for communicating the machine readable programming instructions to the microprocessor through the receiver.
